(19) United States
(12) Patent Application Publication
(10) Pub. No.: US 2015/0013672 A1
(43) Pub. Date: Jan. 15, 2015

(54) ORAL AIRWAY ADJUNCT AND METHOD FOR MAKING THE SAME

(71) Applicant: Akram Abdoue, Palos Park, IL (US)

(72) Inventor: Akram Abdoue, Palos Park, IL (US)

(21) Appl. No.: 14/326,685

(22) Filed: Jul. 9, 2014

Related U.S. Application Data
(60) Provisional application No. 61/844,081, filed on Jul. 9, 2013.

(51) Int. Cl. A61M 16/04; B29C 67/00

(52) U.S. Cl. CPC A61M 16/0495; B29C 67/0051; A61M 2207/10; B29K 2027/06
USPC 128/200.26; 264/401

(57) ABSTRACT

An oral airway adjunct includes a passageway extending from the proximal end to the distal end, wherein the passageway is sized to pass through a breathing tube. The oral airway adjunct also includes a flange located at the proximal region and a curved region extending away from the proximal region towards the distal region. The curved region is defined by a varying radius.

### CROSS REFERENCE TO RELATED APPLICATIONS

This application claims priority to U.S. Patent Application No. 61/844,081, filed on Jul. 9, 2013, the entirety of which is herein incorporated by reference.

### FEDERALLY SPONSORED RESEARCH OR DEVELOPMENT

[Not Applicable]

### JOINT RESEARCH AGREEMENT

[Not Applicable]

### SEQUENCE LISTING

[Not Applicable]

### BACKGROUND

The present technology relates to an oral airway adjunct that is customizable for a patient.

An oral airway adjunct (also known as oropharyngeal airway, an oral airway, OPA, or a Guedel pattern airway) is a medical device used to maintain an open airway in a patient. The oral airway adjunct may prevent the patient's tongue from covering the epiglottis, which could prevent the person from breathing without obstruction. When a patient becomes unconscious, the muscles in the jaw relax and allow the tongue to obstruct the airway.

An oral airway adjunct may include an elongated and generally curved tube that is inserted into the mouth of the user and over the tongue. The oral airway adjunct, once positioned, creates an air passageway between the mouth and the posterior pharyngeal wall. This may be useful when the tongue and/or the epiglottis fall backwards against the posterior pharynx in patients that are anesthetized or unconscious.

The oral airway adjunct may be used to position a breathing tube (e.g., endotracheal tube) in the patient. The endotracheal tube can be passed through a passageway in the oral airway adjunct and down into the trachea.

Patient anatomies vary. For example, some patients may have a small mandible, some patients may have a larger tongue, and other patients may have redundant tissues that occlude the airway during general anesthesia. As a result of these different airway anatomies, insertion of oral airway adjuncts in patients can be a difficult task.

### SUMMARY

According to certain inventive techniques, an oral airway adjunct includes a proximal end, a distal end, a proximal region, a distal region, a superior side, and an inferior side. The oral airway adjunct includes a passageway, a flange, and a curved region. The passageway extends from the proximal end to the distal end. The passageway is sized to pass through a breathing tube. The flange is located at the proximal region. A curved region extends away from the proximal region towards the distal region. The curved region is defined by a varying radius. The axial cross-section of the passageway may be constant along the length of the passageway. Alternatively, the axial cross-section of the passageway may narrow in the distal region, thereby defining a ramp in the passageway. The ramp may extend away from the superior side towards the inferior side. The distal end may have a bevel. The long end of the bevel may be located on the inferior side. The short end of the bevel may be located on the superior side. The oral airway adjunct may be formed with a material comprising polyvinyl chloride (PVC).

According to certain inventive techniques, an oral airway adjunct includes a proximal end, a distal end, a proximal region, a distal region, a superior side, and an inferior side. The oral airway adjunct includes a passageway, a flange, and a curved region. The passageway extends from the proximal end to the distal end. The passageway is sized to pass through a breathing tube. The flange is located at the proximal region. A curved region extends away from the proximal region towards the distal region. The curved region may be defined by a varying radius. The axial cross-section of the passageway narrows in the distal region, thereby defining a ramp in the passageway. The ramp may extend away from the superior side towards the inferior side. The distal end may have a bevel. The long end of the bevel may be located on the inferior side. The short end of the bevel may be located on the superior side. The oral airway adjunct may be formed with a material comprising PVC.

According to certain inventive techniques, a method for creating an oral airway adjunct for use in an airway of a patient includes: receiving an image of the patient's airway; and sizing a virtual oral airway adjunct according to the image. The method may also include constructing the oral airway adjunct according to the virtual oral airway adjunct. This may involve three-dimensional printing. The oral airway adjunct may include a proximal end, a distal end, a proximal region, a distal region, a superior side, and an inferior side. The oral airway adjunct may include a passageway, a flange, and a curved region. The passageway extends from the proximal end to the distal end. The passageway is sized to pass through a breathing tube. The flange is located at the proximal region. A curved region extends away from the proximal region towards the distal region. The curved region is defined by a varying radius. The axial cross-section of the passageway may be constant along the length of the passageway. Alternatively, the axial cross-section of the passageway may narrow in the distal region, thereby defining a ramp in the passageway. The ramp may extend away from the superior side towards the inferior side. The distal end may have a bevel. The long end of the bevel may be located on the inferior side. The short end of the bevel may be located on the superior side. The oral airway adjunct may be formed with a material comprising PVC.

### BRIEF DESCRIPTION OF SEVERAL VIEWS OF THE DRAWINGS

FIG. 1A illustrates a perspective view of an oral airway adjunct, according to the prior art.

FIG. 1B illustrates an elevational view of an oral airway adjunct, according to the prior art.

FIGS. 2A and 2B illustrate elevational views of an oral airway adjunct, according to certain inventive techniques.

FIG. 2C illustrates a longitudinal cross-sectional view of an oral airway adjunct, according to certain inventive techniques.

FIGS. 3A and 3B illustrate elevational views of an oral airway adjunct, according to certain inventive techniques.

FIG. 3C illustrates a longitudinal cross-sectional view of an oral airway adjunct, a according to certain inventive techniques.

FIG. 4 illustrates a flowchart for a method of constructing an oral airway adjunct, according to certain inventive techniques.

FIGS. 5A and 5B illustrate a virtual oral airway adjunct in a patient's airway, according to certain inventive techniques.

FIGS. 6A-6C illustrate a method of constructing an oral airway adjunct, according to certain inventive techniques.

The foregoing summary, as well as the following detailed description of certain techniques of the present application, will be better understood when read in conjunction with the appended drawings. For the purposes of illustration, certain techniques are shown in the drawings. It should be understood, however, that the claims are not limited to the arrangements and instrumentality shown in the attached drawings.

### DETAILED DESCRIPTION

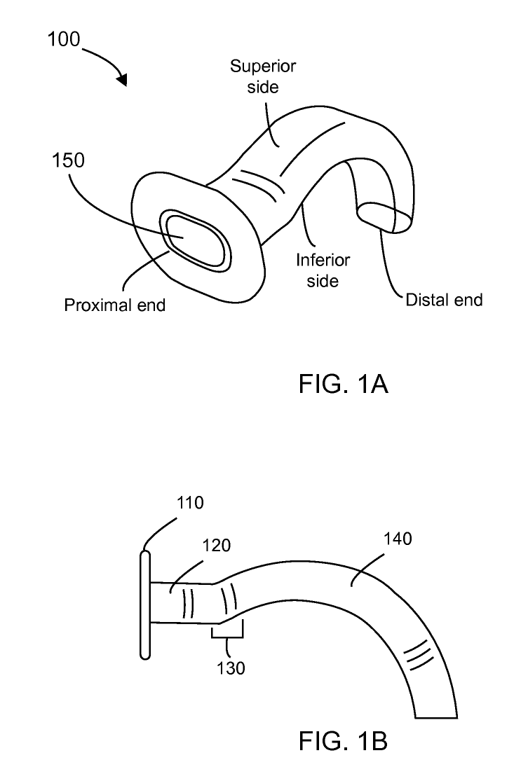

FIG. 1A illustrates a perspective view of an oral airway adjunct 100, according to the prior art. FIG. 1B illustrates an elevational view of the oral airway adjunct 100.

The oral airway adjunct 100 has a proximal end and a distal end. It also has a superior side and an inferior side. The oral airway adjunct 100 may include a flange 110, a straight region 120, a transition region 130, a curved region 140, and a passageway 150. The flange 110 may be located in a proximal region of the oral airway adjunct 100. The straight region 120 may extend distally from the flange 110. A transition region 130 may extend distally from the straight region 120. The curved region 140 may extend distally from the transition region 130. When viewed from the inferior side, the transition region 130 may have a convex curvature, and the curved region 140 may have a concave curvature. The curvature of the curved region 140 may be defined by a constant radius. A passageway 150 may extend from the proximal end to the distal end of the oral airway adjunct 100. The passageway 150 may be sized to receive a breathing tube that assists with breathing.

Because of the uniform shape of the prior art oral airway adjunct 100, it may be necessary to use handles and/or blades to force open the mouth. Such a process may cause trauma to teeth, tongue, lips and/or uvula and that could lead to laceration of lips, broken or dislodged teeth, tongue laceration and/or bleeding of inside of the airway or displacement of the teeth to the trachea and lungs. In some patients with atypical airways, visualization of vocal cords to place the breathing tube may be difficult.

The present technology relates generally to the insertion of a breathing tube inside a patient. For example, the present technology relates to systems and methods that assist in the placement of breathing endotracheal tubes inside patients undergoing general anesthesia or mechanical ventilation.

The present technology provides an oral airway adjunct that is to be placed inside the mouth of a patient, extending from the gum/teeth line to the tracheal opening where the endotracheal tube will be positioned. In certain embodiments of present technology, the oral airway adjunct can operate with a soft and/or flexible endotracheal tube that will allow the breathing tube to pass more easily through the instrument to reach the airway.

The sizes, shapes and other specific anatomical features of an airway can vary from patient to patient. Often times, this variance can be significant. For example, some patients may have a small mandible. Other patients may suffer from sleep apnea, have a larger tongue, and/or have redundant tissues that occlude the airway after the patient has been induced for general anesthesia. Moreover, some patients, for example, trauma patients and/or patients with cervical spine or neck injuries may require strict precautions to keep the cervical spine in a neutral position. Further, some patients may need to be intubated with a breathing tube while awake. In these circumstances, along with many others, insertion of a breathing tube can be a difficult task.

The present technology assists in the placement of a breathing tube inside a patient by providing an oral airway adjunct that conforms to the specific to the anatomy of that particular patient. Such an oral airway adjunct can be used, for example, by an anesthesiologist on the day of surgery and can stay with that patient throughout the course of the patient's visit to the hospital or healthcare facility.

The oral airway adjunct can create a passageway that connects the mouth inlet opening to the trachea. This can allow a breathing tube to slide easily through the oral airway adjunct and positioned inside of the tracheal lumen, which can be used for mechanical ventilation.

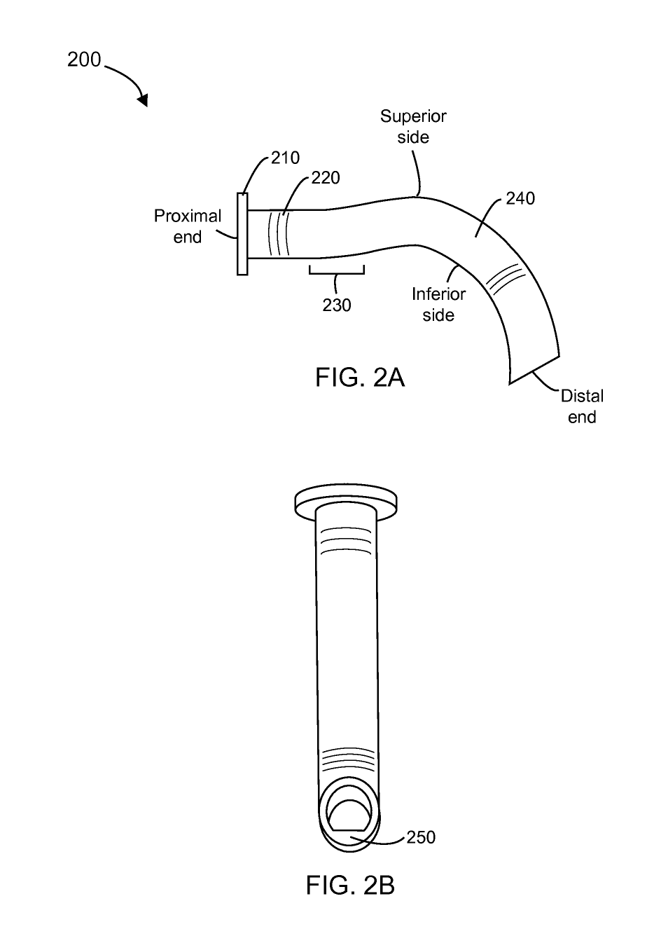

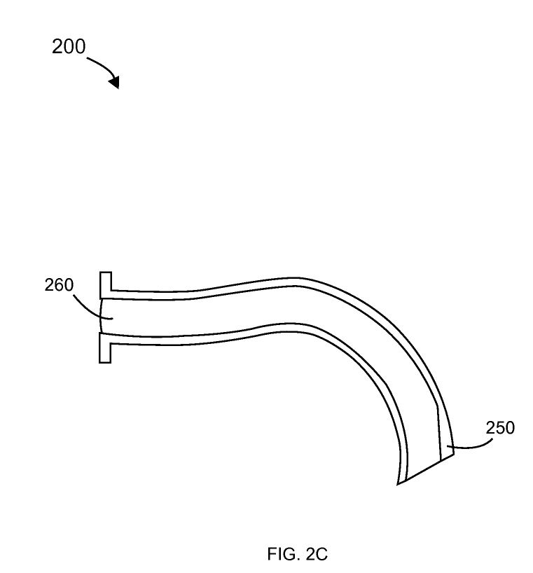

FIG. 2A illustrates a perspective view of an oral airway adjunct 200, according to certain inventive techniques. FIG. 2B illustrates an elevational view of the oral airway adjunct 200. FIG. 2C illustrates a longitudinal cross-sectional view of the oral airway adjunct 200.

The oral airway adjunct 200 has a proximal end and a distal end. It also has a superior side and an inferior side. The oral airway adjunct 200 may include a flange 210, a straight region 220, a transition region 230, a curved region 240, and a passageway 260. The flange 210 may be located in a proximal region of the oral airway adjunct 200. The straight region 220 may extend distally from the flange 210. A transition region 230 may extend distally from the straight region 220. The curved region 240 may extend distally from the transition region 230. When viewed from the inferior side, the transition region 230 may have a convex curvature, and the curved region 240 may have a concave curvature. The curvature of the curved region 240 may be defined by a varying radius. A passageway 250 may extend from the proximal end to the distal end of the oral airway adjunct 200. The passageway 250 may be sized to receive a breathing tube that assists with breathing.

An axial cross-section of the passageway 260 may be constant along the length of the passageway 260. As another option, the axial cross-section of the passageway 260 may narrow in a region near the distal end. This narrowing can form a ramp 250. The ramp 250 may guide the breathing tube upon its exit from the passageway 260 into the trachea. The ramp 250 may extend away from the superior side and towards the inferior side of the oral airway adjunct 200. After placement of the oral airway adjunct 200, the breathing tube may be advanced through the passageway 250 until it passes the distal opening and goes through the vocal cords to the inside of the tracheal lumen. The axial cross-section of the passageway 260 may be circular, rectangular (for example, with rounded corners), ovular, or the like.

The distal end of the oral airway adjunct 200 may have a bevel. The long end of the bevel may be located on the inferior side, and the short end of the bevel may be located on the superior side of the oral airway adjunct 200.

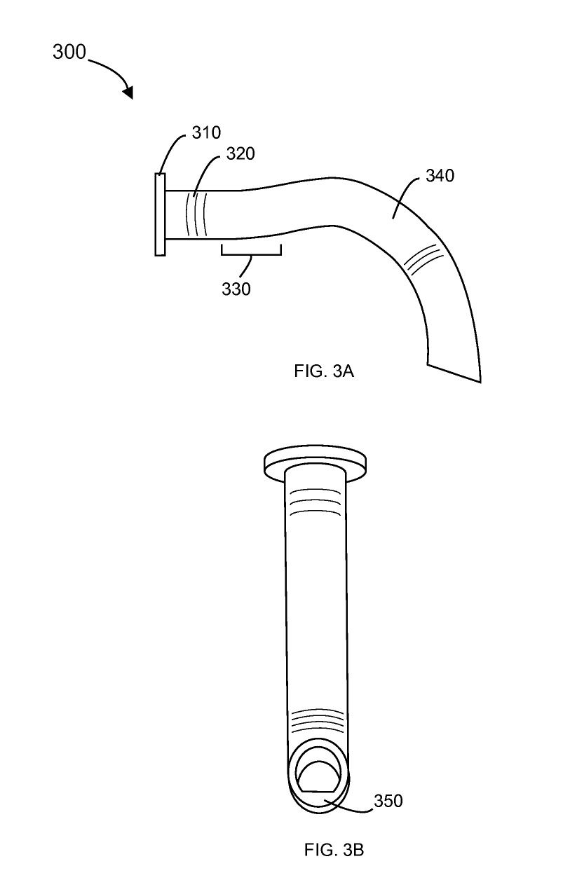

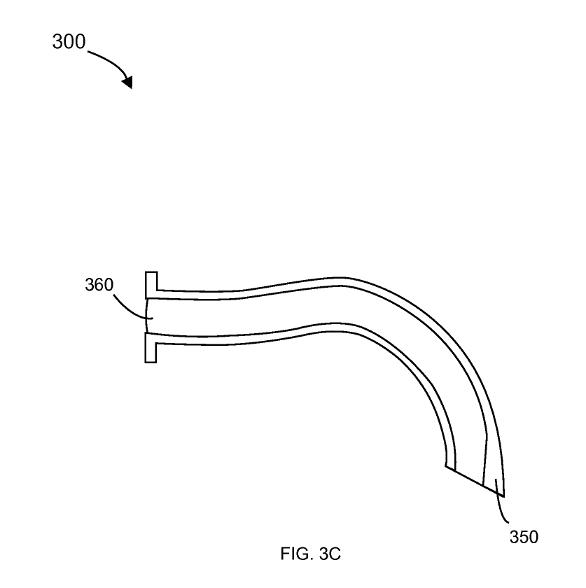

FIG. 3A illustrates a perspective view of an oral airway adjunct 300, according to certain inventive techniques. FIG. 3B illustrates an elevational view of the oral airway adjunct 300. FIG. 3C illustrates a longitudinal cross-sectional view of the oral airway adjunct 300. The oral airway adjunct 300 may be similar to the oral airway adjunct 200, except for the orientation of the bevel. In this embodiment, the long end of the bevel may be located on the superior side, and the short end of the bevel may be located on the inferior side of the oral airway adjunct 300.

The bevel in oral airway adjunct 200 or 300 may guide the endotracheal tube anteriorly to reach the glattic opening and trachea. Without the bevel, there is a chance that the endotracheal tube might slide posteriorly to the esophagus instead.

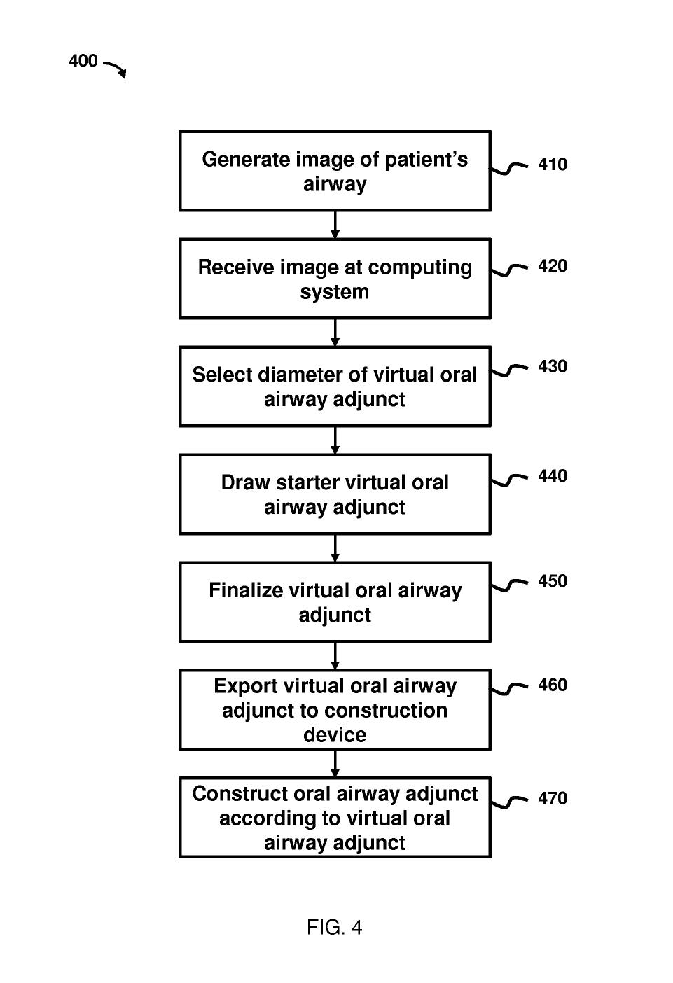

FIG. 4 illustrates a flowchart 400 for a method of creating an oral airway adjunct for use in an airway of a patient, according to certain inventive techniques. The flowchart 400 is shown to have various steps. Some steps may be omitted, and/or may be performed in a different order. Some steps may be performed substantially simultaneously. Flowchart 400, or a portion thereof, may be performable by a computing system including software, hardware, firmware, and/or computer-readable storage media. The computing system may have one or more processors. Flowchart 400, or a portion thereof, may also be expressible through a set of instructions (for execution on one or more processors) stored on one of more computer-readable storage media, such as RAM, ROM, EPROM, EEPROM, optical disk, magnetic disk, magnetic tape, and/or the like.

At step 410, an image of a patient's airway may be generated. Such an image may be a two or three dimensional image. The image may be generated by a CT scan, MRI, X-ray, ultrasound, or another medical imaging modality. The image may be a digital image. At step 420, the image of the patient's airway is received at a computing system, for example, through a network connection. A user is capable of interacting with the computing system through user input devices (e.g., mouse and/or keyboard). Signals from the user input devices are received by at least one processor.

At step 430, the user may select a property of a virtual oral airway adjunct. Such properties include outer diameter, diameter of the passageway, material to be used, or the like. At step 440, a starter virtual oral airway adjunct may be sized (e.g., drawn), either automatically or by the user through the user input devices. The starter virtual oral airway adjunct may be a tube, for example, without a flange, ramp, and/or bevel. At step 450, additional features may be added to the starter virtual oral airway adjunct to form a virtual oral airway adjunct. Such features may include the flange, ramp, and/or bevel. The user may be able to customize these features, for example: size of the flange, angle of the bevel, and/or length and height of the ramp.

At step 460, the virtual oral airway adjunct may be exported to a construction device, such as a three-dimensional printer. At step 470, the oral airway adjunct may be constructed (for example, three-dimensionally printed) according to the virtual oral airway adjunct.

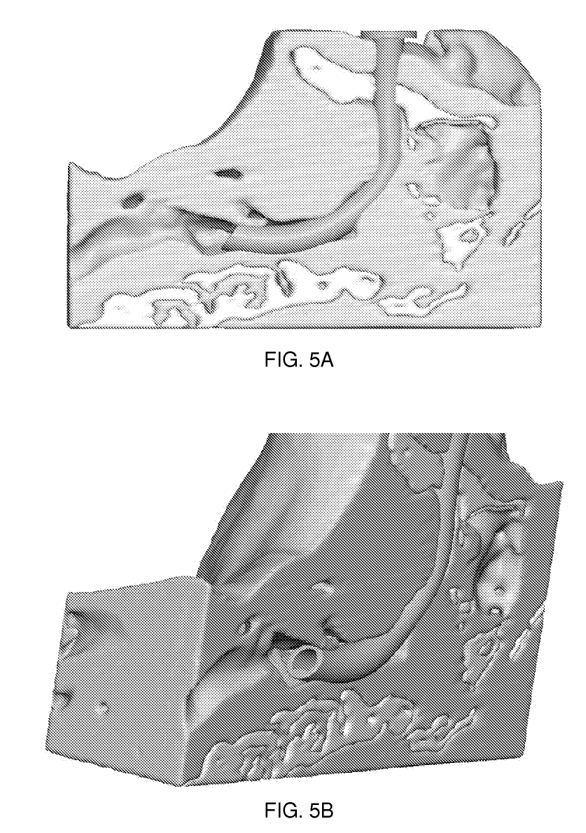

FIGS. 5A and 5B illustrate a virtual oral airway adjunct in a patient's airway, according to certain inventive techniques.

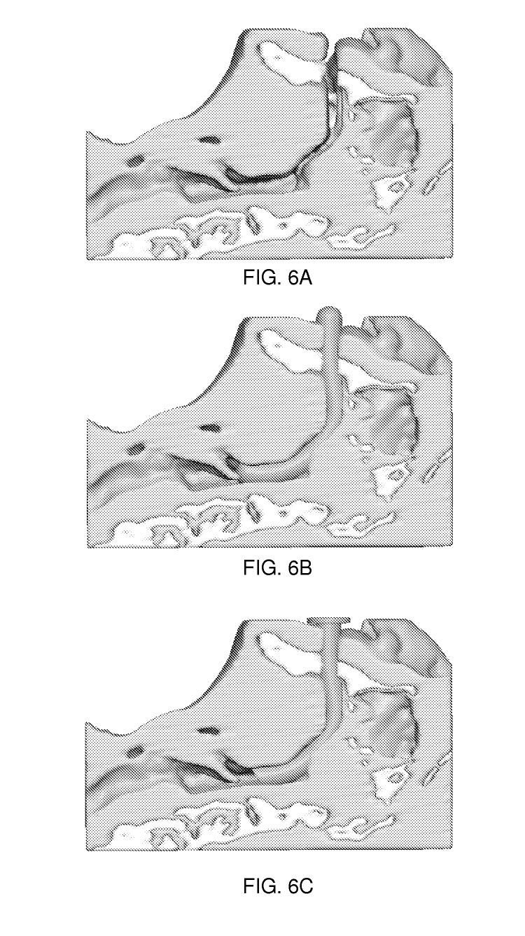

FIGS. 6A-6C illustrate a method of constructing an oral airway adjunct, according to certain inventive techniques. FIG. 6A illustrates an image of the patient's airway. FIG. 6B shows a starter virtual oral airway adjunct in the image. FIG. 6C illustrates a finalized virtual oral airway adjunct (including the flange and bevel) in the image of the patient's airway. The virtual oral airway adjunct can now be exported for construction (for example, with a three-dimensional printer).

Aspects of the techniques described herein may be implemented in digital electronic circuitry, computer software, firmware, or hardware, including the structures disclosed herein and their structural equivalents, or in various combinations. Aspects of the techniques described herein may be implemented as one or more computer programs, for example, one or more sets of program instructions residing on or encoded in a computer-readable storage medium for execution by, or to control the operation of, one or more processors. Alternatively or in addition, the instructions may be encoded on an artificially-generated propagated signal, for example, a machine-generated electrical, optical, or electromagnetic signal that may be generated to encode information for transmission to a suitable receiver apparatus for execution by one or more processors. A computer-readable medium may be, or be included in, a computer-readable storage device, a computer-readable storage substrate, a random or serial access memory array or device, various combinations thereof. Moreover, while a computer-readable medium may or may not be a propagated signal, a computer-readable medium may be a source or destination of program instructions encoded in an artificially-generated propagated signal. The computer-readable medium may also be, or be included in, one or more separate physical components or media (for example, CDs, disks, or other storage devices).

Certain techniques described in this specification may be implemented as operations performed by one or more processors on data stored on one or more computer-readable mediums or received from other sources. The term “processor” may encompass various kinds of apparatuses, devices, or machines for processing data, including by way of example a central processing unit, a microprocessor, a microcontroller, a digital-signal processor, programmable processor, a computer, a system on a chip, or various combinations thereof. The processor may include special purpose logic circuitry, for example, a field programmable gate array or an application-specific integrated circuit.

Program instructions (for example, a program, software, software application, script, or code) may be written in various programming languages, including compiled or interpreted languages, declarative or procedural languages, and may be deployed in various forms, for example as a stand-alone program or as a module, component, subroutine, object, or other unit suitable for use in a computing environment. Program instructions may correspond to a file in a file system. Program instructions may be stored in a portion of a file that holds other programs or data (for example, one or more scripts stored in a markup language document), in a dedicated file or in multiple coordinated files (for example, files that store one or more modules, sub-programs, or portions of code). Program instructions may be deployed to be executed on one or more processors located at one site or distributed across multiple sites connected by a network.

It will be understood by those skilled in the art that various changes may be made and equivalents may be substituted without departing from the scope of the novel techniques disclosed in this application. In addition, many modifications may be made to adapt a particular situation or material to the teachings of the novel techniques without departing from its scope. Therefore, it is intended that the novel techniques not be limited to the particular techniques disclosed, but that they will include all techniques falling within the scope of the appended claims.

1. An oral airway adjunct including a proximal end, a distal end, a proximal region, a distal region, a superior side, and an inferior side, wherein the oral airway adjunct comprises:
a passageway extending from the proximal end to the distal end, wherein the passageway is sized to pass through a breathing tube;
a flange located at the proximal region; and
a curved region extending away from the proximal region towards the distal region,
wherein the curved region is defined by a varying radius.

2. The oral airway adjunct of claim 1, wherein an axial cross-section of the passageway is constant along the length of the passageway.

3. The oral airway adjunct of claim 1, wherein an axial cross-section of the passageway narrows in the distal region, thereby defining a ramp in the passageway.

4. The oral airway adjunct of claim 3, wherein the ramp extends away from the superior side towards the inferior side.

5. The oral airway adjunct of claim 3, wherein the distal end comprises a bevel.

6. The oral airway adjunct of claim 5, wherein:
a long end of the bevel is located on the inferior side; and
a short end of the bevel is located on the superior side.

7. The oral airway adjunct of claim 1, wherein the distal end comprises a bevel.

8. The oral airway adjunct of claim 7, wherein:
a long end of the bevel is located on the inferior side; and
a short end of the bevel is located on the superior side.

9. The oral airway adjunct of claim 1, wherein the oral airway adjunct is formed with a material comprising polyvinyl chloride (PVC).

10. A method for creating an oral airway adjunct for use in an airway of a patient, wherein the method comprises:
receiving an image of the patient's airway;
sizing a virtual oral airway adjunct according to the image.

11. The method of claim 10, further comprising constructing the oral airway adjunct according to the virtual oral airway adjunct.

12. The method of claim 11, wherein said constructing the oral airway adjunct comprises three-dimensional printing.

13. The method of claim 11, wherein the oral airway adjunct includes a proximal end, a distal end, a proximal region, and a distal region, wherein the oral airway adjunct comprises:
an passageway extending from the proximal end to the distal end, wherein the passageway is sized to pass through a breathing tube;
a flange located at the proximal region; and
a curved region extending away from the proximal region towards the distal region,
wherein the curved region is defined by a varying radius.

14. The method of claim 13, wherein the passageway has a constant axial cross-section along the length of the passageway.

15. The method of claim 13, wherein the passageway has a narrowing axial cross-section in the distal region, thereby defining a ramp in the passageway.

16. The method of claim 15, wherein the distal end comprises a bevel.

17. The method of claim 13, wherein the distal end comprises a bevel.

18. The method of claim 13, wherein the oral airway adjunct is formed with a material comprising polyvinyl chloride (PVC).

19. An oral airway adjunct including a proximal end, a distal end, a proximal region, a distal region, a superior side, and an inferior side, wherein the oral airway adjunct comprises:
a passageway extending from the proximal end to the distal end, wherein the passageway is sized to pass through a breathing tube;
a flange located at the proximal region; and
a curved region extending away from the proximal region towards the distal region,
wherein an axial cross-section of the passageway narrows in the distal region, thereby defining a ramp in the passageway.

20. The oral airway adjunct of claim 19, wherein the ramp extends away from the superior side towards the inferior side.
